(19) United States
(12) Patent Application Publication
(10) Pub. No.: US 2019/0378618 A1
(43) Pub. Date: Dec. 12, 2019

(54) Machine Learning Systems For Surgery Prediction and Insurer Utilization Review

(71) Applicant: Daniel M. Lieberman, Phoenix, AZ (US)

(72) Inventor: Daniel M. Lieberman, Phoenix, AZ (US)

(21) Appl. No.: 16/004,205

(22) Filed: Jun. 8, 2018

(51) Int. Cl. G16H 50/20; G06N 3/08; G06N 5/04; G06N 99/00; G16H 10/20; G16H 20/40; G06Q 40/08

(52) U.S. Cl. CPC G16H 50/20; G06N 3/08; G06N 5/04; G06Q 40/08; G16H 10/20; G16H 20/40; G06N 99/005

(57) ABSTRACT

Systems and methods are disclosed for surgery prediction. One method includes receiving, from a patient interacting with a survey user interface, a set of survey results, then applying at least one previously trained machine learning model (e.g., one or more artificial neural networks) to the survey results to generate a prediction output. The prediction output includes (i) a first confidence level associated with whether the patient is a surgical candidate for a particular surgical procedure; and, optionally, (ii) a set of second confidence levels associated with a respective set of surgical outcomes. Such systems and methods may be used, for example, by surgeons, health care providers, and insurers performing utilization review.

### TECHNICAL FIELD

The present invention relates, generally, to systems and methods for determining whether a patient requires a surgical procedure and, more particularly, to techniques for determining whether specific surgeries are required in the context of, for example, insurer utilization reviews.

### BACKGROUND

Determining whether a patient is a surgical candidate and, in particular, which surgery should be performed, can be challenging. Such determinations are important, however, as they have a profound impact on patient health, healthcare costs, and other individual and societal factors.

In the context of healthcare insurance providers, it is particularly desirable to avoid false-positives—i.e., instances in which a patient is incorrectly classified as a surgical candidate and/or subjected to unnecessary surgical procedures. Toward that end, health insurance providers often carry out a “utilization review” in which the insurer evaluates the medical necessity of a requested medical procedure for the purpose of providing preauthorization.

Even given recent advances in surgical procedures, insurance case management techniques, and data analysis, healthcare costs (and consequently insurance premiums) continue to rise in an unsustainable fashion. This is due in part to the difficulty in determining whether a patient is a surgical candidate and, if so, which surgical procedure or procedures are a medical necessity.

Systems and methods are thus needed which overcome the limitations of the prior art. Various features and characteristics will also become apparent from the subsequent detailed description and the appended claims, taken in conjunction with the accompanying drawings and this background section.

### BRIEF SUMMARY

Various embodiments of the present invention relate to systems and methods for, inter alia: i) using machine learning techniques and patient survey results to determine whether a patient is a candidate for a particular surgical procedure; ii) improving insurer utilization reviews using the machine learning systems described herein; iii) using multiple pre-trained artificial neural networks to implement the machine learning systems described herein; and iv) using the machine learning systems described herein to improve spine surgery recommendations.

Various other embodiments, aspects, and features are described in greater detail below.

### BRIEF DESCRIPTION OF THE DRAWING FIGURES

Exemplary embodiments will hereinafter be described in conjunction with the following drawing figures, wherein like numerals denote like elements, and:

FIG. 1 is a schematic overview of a system for generating survey responses in accordance with various embodiments;

FIG. 2 is a schematic flow diagram of a machine learning system for surgery prediction in accordance with various embodiments;

FIG. 3 is a schematic block diagram of a shallow artificial neural network (ANN) in accordance with various embodiments;

FIG. 4 is a schematic block diagram of a probabilistic neural network (PNN) in accordance with various embodiments; and

FIG. 5 is a dataflow diagram illustrating an insurance utilization review process in accordance with various embodiments.

### DETAILED DESCRIPTION

The following detailed description of the invention is merely exemplary in nature and is not intended to limit the invention or the application and uses of the invention. Furthermore, there is no intention to be bound by any theory presented in the preceding background or the following detailed description.

Various embodiments of the present invention relate to systems and methods for applying machine learning techniques to surgery prediction—i.e., determining whether a patient is a surgical candidate along with surgical outcome associated with one or more types of surgery. Such systems and methods are particularly advantageous for insurance providers performing utilization review, but may also be used by health care providers, surgeons, and any other party for whom accurate surgery prediction is important.

In general, an exemplary method of surgical prediction includes receiving, from a patient interacting with a survey user interface, a set of survey results, then applying at least one previously trained machine learning model (e.g., one or more artificial neural networks) to the survey results to generate a prediction output. The prediction output includes (i) a first confidence level associated with whether the patient is a surgical candidate for a particular procedure; and (ii) a set of second confidence levels associated with a respective set of outcomes. These outcomes may include, for example, patient satisfaction, complication rate, cost, freedom from pain, improved function, and optimal facility.

In some embodiments, the surgery is considered appropriate if it falls within an acceptable degree of similar cases in a training data set. That is, the training data set will generally include a collection of cases previously determined to be appropriate by an expert panel. In a hip replacement scenario, for example, the clinical decision for surgery is made on the history (survey); past medical history; physical exam; and x ray. In various embodiments, the system is trained by collecting this data from patients who are considered appropriate for surgery by an expert panel. The size of the training set may vary depending upon a variety of factors, but in some embodiments may be greater than 10,000 cases, e.g., approximately 50,000 cases. Subsequently, the surgeon may submit some CPT codes indicting the proposed surgery, and some ICDio codes showing the diagnosis. The insurer or other entity may then collect the clinicals and run the trained model to determine if the proposed surgery is appropriate.

As the system collects data on appropriate cases, it will also have the opportunity to collect the outcome data, such as complication rates, patient satisfaction, willingness to do it again, need for pain medicine, and facility scores, all of which can be generated as associated class data.

While systems and methods are often described herein in the context of surgery and surgical procedures, the invention is not so limited, and may be used to predict the necessity of a wide range of invasive and non-invasive treatments. The phrase “surgery” is thus used herein without loss of generality.

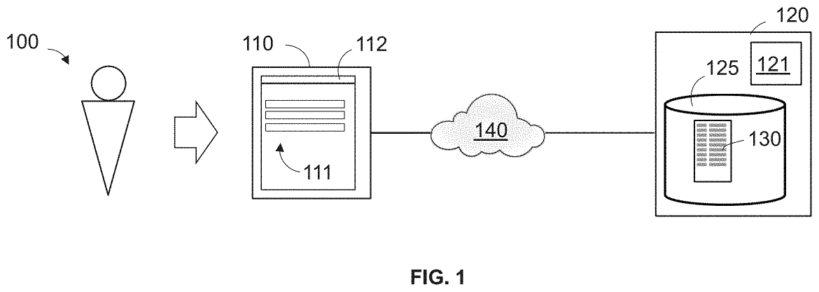

FIG. 1 is a schematic overview of a system for generating survey responses in accordance with various embodiments. More particularly, in this web-based example, a patient 100 interacts with a survey user interface 112 displayed via a computing device no (e.g., a desktop computer, laptop computer, tablet device, smart-phone, or the like). Survey user interface 112 includes a series of questions or prompts 111 configured to elicit responses from patient 100. These responses may take a variety of forms, for example, short answer (text), yes/no selection (Boolean), numeric values (integer or floating point), voice recordings, images, and biometric input data. Responses may be selected and/or entered using a variety of user interface elements known in the art, such as radio buttons, drop-down menus, text entry boxes, buttons, and date fields. As described in further detail below, survey questions 211 may relate to, for example, basic patient information (age, weight, height, etc.), past treatments, exercise level, past accidents, current symptoms, pain levels, and other such questions that might be used as input to a machine learning system.

In the illustrated embodiment, user interface 112 is a web page or collection of web pages displayed in a web browser operating on device no and provided by a survey module 121 (e.g., a web service with associated back end databases, software, etc.) located at a remote server 120. Server 120 may be associated with, for example, an insurance provider, a health care provider, or an individual surgeon.

Interaction of patient 100 with survey user interface 112 causes survey results 130 to be generated and transmitted over network 140 (e.g., the Internet) to server 120, whereupon they are stored within a database 125. Survey results 130 are preferably transmitted in a secure fashion, e.g., via an https protocol.

In some embodiments, data entered by patient 100 may be transformed to produce survey results 130 that are better configured for use by a machine learning system. For example, one of the questions 111 may be an open-ended question such as, “how would you describe your back pain right now.” In response, the patient may be asked to type (or speak) a response, which is then provided to a speech recognition system and/or natural language processing system (e.g., within server 120 or accessible via network 140) to convert the text or spoken utterance to an integer value from 0 to 10.

It will be appreciated that the particular architecture illustrated in FIG. 1 is not intended to be limiting. The components of the illustrated system (e.g., database 125, module 121, and server 120) may be distributed between multiple remote locations and parties. Furthermore, survey user interface 112 need not be implemented as a web-based system, but might be a stand-alone program running on device no that stores survey results 130 locally for later transmission and processing. In some embodiments, survey user interface 112 is an application that can be downloaded to device no via a publicly accessible app store.

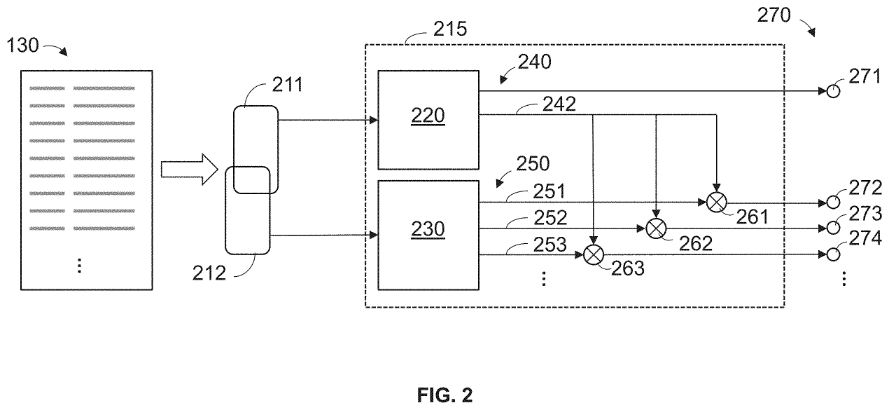

FIG. 2 is a schematic flow diagram illustrating a machine learning system for surgery prediction in accordance with various embodiments. In general, a machine learning module (or simply “module”) 215 is configured to receive survey inputs (e.g., survey inputs 211 and 212) derived from the survey results 130 previously generated (in FIG. 1) by patient 100 in conjunction with the survey module 121. As shown, the survey inputs to model 205 may take the form of multiple sets of inputs (211, 212), each of which is a subset of survey results 130. In the illustrated embodiment, machine learning model 220 receives survey inputs 231, and machine learning model 230 receives survey inputs 232.

In various embodiments, depending upon the nature of the surgery and the types of machine learning techniques being implemented, survey inputs 211 and 212 may be identical, disjoint, or may overlap to some extent. As described in further detail below, each of the inputs 231 and 232 are typically one-dimensional vectors or lists of values with diverse types (e.g., Boolean, floating point, integer, text, etc.).

Module 215 is configured to apply at least one previously trained machine learning model (e.g., machine learning models 220 and 230) to the received survey results 130 to produce a prediction output 270 comprising a series of individual outputs 271, 272, 273, and 274. In one embodiment, output 271 is a confidence level (i.e., a probability value in the range 0.0-1.0) associated with whether the patient 100 is or is not a surgical candidate, and outputs 272-274 are a set of confidence levels associated with a set of corresponding surgical procedures.

In various embodiments, machine learning model 220 is a classifier trained to determine the binary question of whether the patient 100 is a surgery candidate (e.g., for a particular requested surgical procedure), and machine learning model 230 is a classier trained to determine outcomes associated with a number of various surgical procedures.

More particularly, in one embodiment, machine learning model 220 produces an intermediate output 240 comprising two values (271, 242), wherein output 242 is a confidence level that the patient is a surgical candidate, and output 271 is a confidence level that the patient is a non-surgical candidate. In general, these two values should sum to 1.0. Similarly, machine learning model 230 produces an intermediate output 250 comprising three values: 251, 252, and 253, which are then respectively combined (for example, using respective multiplication elements 261, 262, and 263) with output 242 to provide the final output 270 (outputs 271, 272, 273, and 274). Because output 242 is a confidence level that the patient is a surgical candidate, and each of the outputs 251-253 correspond to confidence levels for various surgical outcomes, the final outputs 272, 273, and 274 are each combined probabilities associated with whether a given surgical procedure is required.

Thus, for example, output 240 (values 271 and 242) may be a vector [0.2, 0.8] (indicating that the patient is likely a surgery candidate) while output 250 may be a vector [0.1, 0.2, 0.7] (corresponding, for example, to respective surgical outcomes “s1”, “s2”, and “s3”). In such a case, the final output (values 271-274) would be [0.2, 0.08, 0.16, 0.56], corresponding to a case in which machine learning model 215 indicates surgery is likely required, and the highest surgical outcome is “s3”.

Empirical testing of machine learning systems in accordance with the present subject matter has shown that such systems exhibit predictive accuracies that meet and often exceed those of providers utilizing heuristic and other traditional techniques.

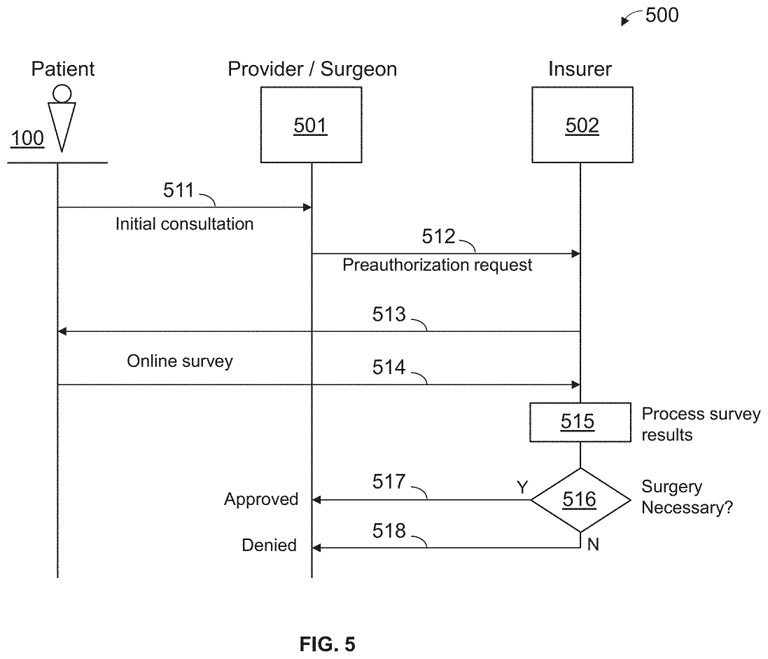

The machine learning system of FIG. 2 may be advantageously utilized by a number of parties, including individual surgeons, healthcare providers, and healthcare insurance companies. FIG. 5, for example, is a conceptual dataflow diagram illustrating one example in which the machine learning module 215 is used in the context of a utilization review performed by a healthcare insurance company. While FIG. 5 illustrates a prospective assessment (i.e., determining whether a treatment is necessary before it is rendered), the methods described herein may also be used in connection with retrospective assessments.

More particularly, referring to FIG. 5, an exemplary utilization review process proceeds as follows. First, patient 100 consults (511) with the healthcare provider or surgeon 501 (generally, the “provider”). As a result of this consultation, the provider 501 and patient 100 have agreed that patient 100 is a surgical candidate, and that a particular surgical procedure should be performed.

Next, a preauthorization request (512) is sent by provider 501 to insurer 502. This request may be accompanied by additional data relevant to the preauthorization requests, such as x-rays, lab results, etc. Insurer may represent any entity configured to cover healthcare costs for patient 100, including private insurers, government payers, health maintenance organizations (HMOs), and self-insured employers.

Insurer 502 then requests (513) that patient 100 fill out an online survey. The source of the survey (e.g., the location of the software code and related database) may be a variety of entities and locations, such as on a server administered by insurer 502. The survey results (514) are then sent by or otherwise collected from patient 100 so that they can be assessed by insurer 502.

Insurer 502 processes the survey results (515) using the various machine learning systems and methods described above, to make a determination as to whether the requested surgical procedure is a required (516). Depending upon this determination, insurer 502 either approves (517) or denies (518) the preauthorization request 512.

In some embodiment, additional machine learning systems are employed to assist in making the determination at 515. For example, in connection with preauthorization request 512, certain lab and test data may also be communicated to insurer. That data itself may be processed to increase prediction accuracy. In one embodiment relating to predicting whether total hip replacement is necessary, insurer receives x-ray images (or raw x-ray data) of relevant anatomical features from provider 501. A previously trained convolutional network (CNN) can utilize the x-ray data as input and produce an output comprising a confidence level, based on the x-ray data alone, that total hip replacement is necessary. That output can be combined with the output of machine learning module 215 to produce a more accurate prediction. The individual outputs may be combined in a variety of ways, using any of the machine learning techniques described herein.

Referring again to FIG. 2, it will be appreciated that machine learning models 220 and 230 may be implemented using a variety of machine learning model types. In one embodiment, for example, machine learning model 220 is implemented as a “shallow” artificial neural network (ANN) classifier, and machine learning model 230 is implemented as a probabilistic neural network (PNN). In one embodiment, models 220 and 230 are trained individually (using training data for inputs 211 ad 212). In other embodiments, the two modules are trained together.

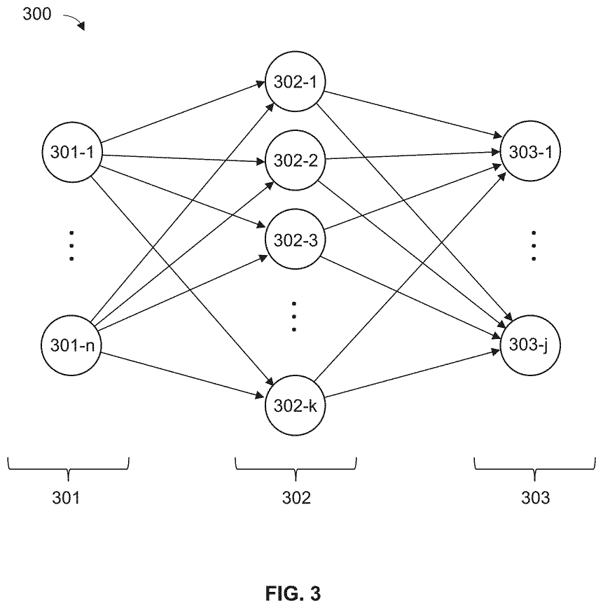

Referring now to FIG. 3 in conjunction with FIGS. 1-2, an example artificial neural network (ANN) implementation 300 that may be used to implement machine learning model 220 will now be described. As shown, ANN 300 includes an input layer 301 with a number of input nodes (e.g., 301-1 to 301-n), an output layer 303 with a number of output nodes (e.g., 303-1 to 303-j), and one or more interconnected hidden layers 302 (in this example, a single hidden layer 302 including nodes 302-1 to 302-k). Thus, inputs 301-1 to 3o1-n correspond to survey inputs 211 of FIG. 2, and outputs 303-1 to 3o3-j correspond to outputs 240 of FIG. 2.

The number of nodes in each layer (n, k, and j) may vary depending upon the application, and in fact may be modified dynamically by the system itself to optimize its performance. In some embodiments (e.g., deep learning systems), multiple hidden layers 302 may be incorporated into ANN 300.

Each of the layers 302 and 303 receives input from a previous layer via a network of weighted connections (illustrated as arrows in FIG. 3). That is, the arrows in FIG. 3 may be represented as a matrix of floating point values representing weights between pairs of interconnected nodes. Each of the nodes implements an “activation function” (e.g., sigmoid, tan h, linear, Relu) that will generally vary depending upon the particular application, and which produces an output that is based on the sum of the inputs at each node.

ANN 300 is trained via a learning rule and “cost function” that are used to modify the weights of the connections in response to the input patterns provided to input layer 301 and the training set provided at output layer 303, thereby allowing ANN 300 to learn by example through a combination of backpropagation and gradient descent optimization. Such learning may be supervised (with known examples of past survey inputs and surgery outcomes provided to input layer 301 and output layer 303), unsupervised (with uncategorized examples provided to input layer 301), or involve reinforcement learning, where some notion of “reward” is provided during training.

Once ANN 300 is trained to a satisfactory level (e.g., without overtraining), it may be used as an analytical tool to make predictions and perform “classification” of the input 301. That is, new inputs are presented to input layer 301, where they are processed by the middle layer 302 and, via forward propagation through the weights associated with each of the edges, produce an output 303. As described above, output layer 303 will typically include a set of confidence levels or probabilities associated with a corresponding number of different classes, such as “non-surgical candidate” and “surgical candidate”.

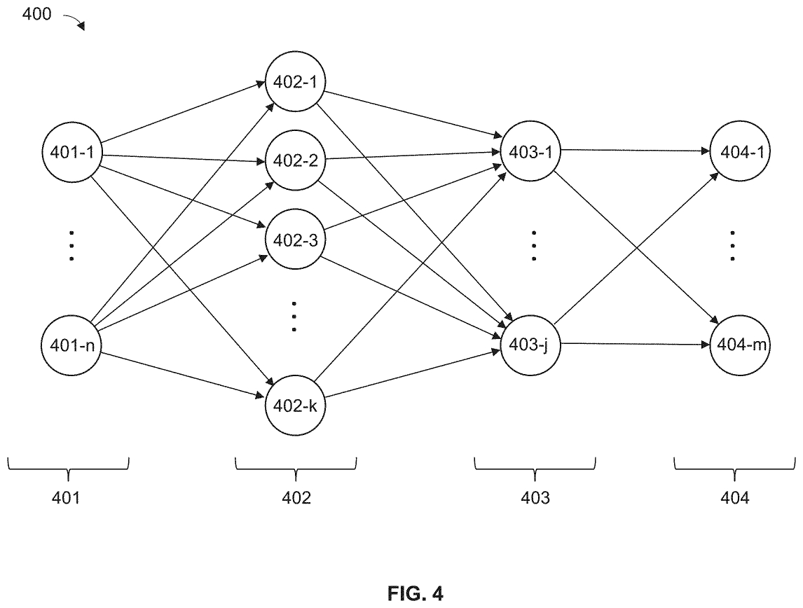

Referring now to FIG. 4 in conjunction with FIGS. 1-2, an example PNN implementation 400 that may be used to implement machine learning model 230 will now be described. A PNN such as PNN 400 is a type of feed-forward neural network based on a Bayesian minimum risk criteria, and is advantageous in that it can be trained quickly and has a relatively simple structure. In general, PNN 400 includes an input layer 401 (including nodes 401-1 to 401-n), pattern layer (or “hidden layer”) 402 (including nodes 402-1 to 402-n), summation layer 403 (including nodes 403-1 to 403-n), and output layer 404 (including nodes 404-1 to 404-n). Thus, inputs 401-1 to 401-n correspond to survey inputs 212 of FIG. 2, and outputs 404-1 to 404-j correspond to outputs 250 of FIG. 2.

As with FIG. 3, the arrows in FIG. 4 represent the interconnections and weights between the various nodes. Each node in the input layer 401 represents a predictor variable, and pattern layer 402 contains one node for each case in the training data set. PNN 400 is trained, as with PNN 300, by applying historical survey inputs to input layer 401 and setting output layer 400 to reflect a successfully selected surgery type corresponding to those past survey inputs. PNN does not require training connection weights, but directly configures hidden layer 402 based on the given training samples (survey inputs 211). In this way, PNN 400 operates in such a way that classifies inputs based on the most similar training data.

While machine learning models 220 and 230 are both described above in the context of artificial neural networks, the range of embodiments are not so limited. Any of the various modules described herein may be implemented as one or more machine learning models that undergo supervised, unsupervised, semi-supervised, or reinforcement learning and perform classification (e.g., binary or multiclass classification), regression, clustering, dimensionality reduction, and/or such tasks. Examples of such models include, without limitation, artificial neural networks (ANN) (such as a recurrent neural networks (RNN) and convolutional neural network (CNN)), decision tree models (such as classification and regression trees (CART)), ensemble learning models (such as boosting, bootstrapped aggregation, gradient boosting machines, and random forests), Bayesian network models (e.g., naive Bayes), principal component analysis (PCA), support vector machines (SVM), clustering models (such as K-nearest-neighbor, K-means, expectation maximization, hierarchical clustering, etc.), linear discriminant analysis models.

The systems and methods described above, such as survey module 121 and machine learning module 215, may be implemented in software using any convenient general-purpose programming language. In one substantially web-based embodiment, the PHP programming language is used in conjunction with standard HTML/CSS/JavaScript techniques. Other suitable languages include, without limitation, web assembly (Wasm), Python, C++, C#, and Java. In addition, various standard machine learning libraries and linear algebra libraries may be employed.

Referring briefly again to FIGS. 1 and 2, it will be apparent that a wide variety of survey questions 111 and survey results 130 may be employed to train machine learning module 215, depending upon the nature of the surgery types involved.

In one embodiment, the machine learning system illustrated in FIGS. 1 and 2 is applied to spine surgery prediction. Common spine treatments include, for example, epidural injection, medial branch block, spinal decompression, spinal fusion, laminectomy (removing the back part of the lamina over the spinal column), microdiscectomy (removing a small portion of a disc to relieve pressure on a nerve), and direct visual rhizotomy (DVR) (cutting nerve root branches that transmit pain signals).

In a particular embodiment, referring to FIG. 2, outputs 272, 273, and 274 correspond, respectively, to confidence levels associated with laminectomy, DVR, and microdiscectomy. In such an embodiment, the survey inputs (and corresponding data types) listed in Table 1 below may be employed. It will be appreciated that this list is not intended to be limiting.

TABLE 1
Example Survey Inputs

| No. | Type | Description |
|---|---|---|
| 1 | float | Age of patient/100 |
| 2 | bool | Treatment: NSAID |
| 3 | bool | Treatment: Chiropractic |
| 4 | bool | Treatment: Epidural |
| 5 | bool | Treatment: Facet/MBB |
| 6 | bool | Treatment: Rhizotomy |
| 7 | bool | Treatment: Surgery |
| 8 | bool | Treatment: Physical Therapy |
| 9 | bool | Symptoms: Leg Numbness |
| 10 | bool | Symptoms: Fecal Incontinence |
| 11 | int | Pain description |
| 12 | int | Pain location |
| 13 | bool | Osteoporosis history |
| 14 | bool | Pain in ankle |
| 15 | bool | Pain while walking |
| 16 | bool | Pain reduced by leaning over cart |
| 17 | bool | Pain worsened by sitting |
| 18 | bool | Pain reduced by leaning forward |
| 19 | bool | Diabetes |

In one embodiment, survey input 211 of FIG. 2 includes, from table 1, inputs 1, 2, 3, 4, 5, 11, 14, 16, 18, and 19; and the survey input 212 includes inputs 1, 3, 5, 6, 7, 8, 9, 10, 12, 13, 15, 17, and 18.

In summary, the present subject matter relates to systems and methods for applying machine learning techniques to surgery prediction—i.e., determining whether a patient is a surgical candidate and, if so, which surgical procedure is likely to be most efficacious. Such systems and methods are particularly advantageous for insurance providers performing utilization review, but may also be used by health care providers, surgeons, and any other party for whom accurate surgery prediction is important.

In some embodiments, the insurer or other party is presented with a set of data relating to the patient along with a proposed surgical procedure. The machine learning model can then determine a probability that the proposed surgical procedure is appropriate or a medical necessity. In other embodiments, a prediction indicating the most appropriate surgery based on the data can also be provided.

Embodiments of the present disclosure may be described herein in terms of functional and/or logical block components and various processing steps. It should be appreciated that such block components may be realized by any number of hardware, software, and/or firmware components configured to perform the specified functions. For example, an embodiment of the present disclosure may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments of the present disclosure may be practiced in conjunction with any number of systems, and that the systems described herein is merely exemplary embodiments of the present disclosure. Further, the connecting lines shown in the various figures contained herein are intended to represent example functional relationships and/or physical couplings between the various elements. It should be noted that many alternative or additional functional relationships or physical connections may be present in an embodiment of the present disclosure.

As used herein, the term “module” refers to any hardware, software, firmware, electronic control component, processing logic, and/or processor device, individually or in any combination, including without limitation: application specific integrated circuits (ASICs), field-programmable gate-arrays (FPGAs), dedicated neural network devices (e.g., Google Tensor Processing Units), electronic circuits, processors (shared, dedicated, or group) configured to execute one or more software or firmware programs, a combinational logic circuit, and/or other suitable components that provide the described functionality.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

1. A machine learning system for surgical prediction, the system comprising:
a survey module configured to generate a survey user interface and receive, from a patient interacting with the survey user interface, a set of survey results;
a machine learning module configured to receive the survey results, apply at least one previously trained machine learning model to the survey results, and produce a prediction output;
wherein the prediction output includes a first confidence level associated with whether the patient is a surgical candidate for a proposed surgical procedure.

2. The system of claim 1, wherein the prediction output further includes a set of second confidence levels associated with a respective set of surgical outcomes.

3. The machine learning system of claim 1, wherein the machine learning module is configured to further receive, and consider in producing the prediction output, at least one of: medical images, past medical history, lab reports, radiology reports.

4. The machine learning system of claim 1, wherein the at least one previously trained machine learning model includes:
a first machine learning model configured to receive a first survey input comprising a first subset of the survey results; and
a second machine learning model configured to receive a second survey input comprising a first subset of the survey results;

5. The machine learning system of claim 4, wherein the first machine learning model is a shallow artificial neural network and the second machine learning model is a probabilistic neural network.

6. The machine learning system of claim 4, wherein the first confidence level is produced by the first machine learning model, and the set of second confidence levels associated with the respective set of surgical outcomes is produced by a combination of an intermediate output of the second machine learning model and the first confidence level.

6. The machine learning system of claim 1, wherein the set of surgical outcomes are associated with spine surgical procedures.

7. The machine learning system of claim 1, wherein:
the survey user interface is configured to receive, in response to at least one survey question, a text input, and;
the survey module is configured to convert the text input, via natural language processing, to a numerical value.

8. A method for performing insurer utilization review comprising:
receiving, at an insurer system, a preauthorization request associated with a patient and a requested treatment;
receiving, from the patient, a set of survey results; and
applying at least one previously trained machine learning model to the survey results to generate a prediction output, wherein the prediction output includes a first confidence level associated with whether the patient is a surgical candidate for a surgical procedure;
selectably denying or approving the preauthorization request based on the requested treatment and the prediction output.

9. The method of claim 8, wherein applying the at least one previously trained machine learning model to the survey results includes:
applying a first subset of the survey results to a first machine learning model; and
applying a second subset of the survey results to a second machine learning model;
wherein the first machine learning model is a shallow artificial neural network and the second machine learning model is a probabilistic neural network.

10. The method of claim 9, wherein:
the prediction output further includes a set of second confidence levels associated with a respective set of surgical outcomes;
the first confidence level is produced by the first machine learning model; and
the set of second confidence levels associated with the respective set of surgical outcomes is produced by a combination of an intermediate output of the second machine learning model and the first confidence level.

11. The method of claim 9, wherein the set of surgical outcomes are associated with spine surgical procedures.

12. The method of claim 11, wherein the spine surgical procedures include laminectomy, direct visual rhizotomy, and microdiscectomy.

13. The method of claim 8, wherein:
the survey user interface is configured to receive, in response to at least one survey question, a text input, and;
the survey module is configured to convert the text input, via natural language processing, to a numerical value.

14. A method for surgical prediction, the method comprising:
training at least one machine learning model based on previously performed surgical procedures;
generating a survey user interface;
receiving, from a patient interacting with the survey user interface, a set of survey results;
applying the at least one previously trained machine learning model to the survey results to produce a prediction output that includes (i) a first confidence level associated with whether the patient is a surgical candidate; and (ii) a set of second confidence levels associated with a respective set of surgical outcomes.

15. The method of claim 14, wherein the at least one previously trained machine learning model includes:
a first machine learning model configured to receive a first survey input comprising a first subset of the survey results; and
a second machine learning model configured to receive a second survey input comprising a first subset of the survey results;

16. The machine learning system of claim 15, wherein the first machine learning model is a shallow artificial neural network and the second machine learning model is a probabilistic neural network.

17. The method of claim 15, wherein the first confidence level is produced by the first machine learning model, and the set of second confidence levels associated with the respective set of surgical outcomes is produced by a combination of an intermediate output of the second machine learning model and the first confidence level.

18. The method of claim 14, wherein the set of surgical outcomes are related to spine surgical procedures.

19. The method of claim 18, wherein the spine surgical procedures include laminectomy, direct visual rhizotomy, and microdiscectomy.

20. The method of claim 14, wherein:
the survey user interface is configured to receive, in response to at least one survey question, a text input, and;
the survey module is configured to convert the text input, via natural language processing, to a numerical value.
